(19) United States
(12) Patent Application Publication
(10) Pub. No.: US 2014/0180395 A1
(43) Pub. Date: Jun. 26, 2014

(54) BIOACTIVE SPIRAL COIL COATING

(71) Applicant: THE REGENTS OF THE UNIVERSITY OF CALIFORNIA, Oakland, CA (US)

(72) Inventors: Benjamin M. Wu, San Marino, CA (US); Arnold Suwarnasarn, Los Angeles, CA (US); Fernando Vinuela, Los Angeles, CA (US); Ichiro Yuki, Los Angeles, CA (US)

(73) Assignee: THE REGENTS OF THE UNIVERSITY OF CALIFORNIA, Oakland, CA (US)

(21) Appl. No.: 14/148,706

(22) Filed: Jan. 6, 2014

Related U.S. Application Data
(63) Continuation of application No. PCT/US2012/044049, filed on Jun. 25, 2012.
(60) Provisional application No. 61/505,470, filed on Jul. 7, 2011.

(51) Int. Cl. A61F 2/82

(52) U.S. Cl. CPC A61F 2/82
USPC 623/1.15; 427/2.25

(57) ABSTRACT

An endovascular spiral coil coating and methods of making and using the same.

### CROSS-REFERENCE TO RELATED APPLICATIONS

This application is a 35 U.S.C. §111(a) continuation of PCT international application number PCT/US2012/044049 filed on Jun. 25, 2012, incorporated herein by reference in its entirety, which claims the benefit of U.S. provisional patent application Ser. No. 61/505,470 filed on Jul. 7, 2011, incorporated herein by reference in its entirety. Priority is claimed to each of the foregoing applications.

The above-referenced PCT international application was published as PCT International Publication No. WO 2013/006298 on January 10, which publication is incorporated herein by reference in its entirety.

### STATEMENT REGARDING FEDERALLY SPONSORED RESEARCH OR DEVELOPMENT

Not Applicable

### INCORPORATION-BY-REFERENCE OF COMPUTER PROGRAM APPENDIX

Not Applicable

### BACKGROUND OF THE INVENTION

1. Field of the Invention

The present invention relates generally to a bioactive coating on a medical device and methods of making and using the same.

2. Background Discussion

Subarachnoid hemorrhage from intracranial aneurysm rupture remains a devastating disease. Endovascular occlusion of ruptured and unruptured intracranial aneurysms using Guglielmi detachable coil (GDC) technology has recently gained worldwide acceptance as a less-invasive treatment alternative to standard microsurgical clipping. However, critical evaluation of the long-term anatomical results of aneurysms treated with metal coils shows three limitations. First, compaction and aneurysm recanalization can occur. This technical limitation is more often seen in small aneurysms with wide necks and in large or giant aneurysms. Second, the standard platinum metal coil is relative biological inert. Recent reports of methods to favorably enhance the biological activity of metal coils highlight the increased interest in finding innovative solutions to overcome these present biological limitations of the conventional metal coil system.

Polymeric coatings carrying a bioactive agent have been used to impart bioactivity to implantable devices (e.g., stents). However, currently available bioactive coils are either by 1) coating the bare platinum core with braided PGLA sutures, or 2) inserting PGA sutures in the spiral coils. There is no coil available at this point with direct coating of the polymeric materials. When a polymeric coating is formed on a coil, often times, the grooves of the spiral coil are coated along with the outer surface of the coil, causing the mechanical flexibility to be compromised, which is undesirable. Further, for a spiral coil to be spatially compatible with a vascular lumen in brain, sometimes it is important to limit the diameter of a coil to a certain size since it is constrained by the inner diameter with the microcatheter. Since the braded suture on the surface of the bare platinum coil is space-consuming, the size of the platinum core requires to be small which results in poor mechanical support. The maximum size of the coil one can deliver is 380 μm in outer diameter due to limited size of delivery microcatheter. Outer coatings on a coil can be desirable from a biomaterial-cell interaction perspective, but excessively thick coatings are undesirable.

Therefore, a need exists for improved coils and methods for brain aneurysm therapy.

The embodiments below address the above identified issues and needs.

### SUMMARY OF THE INVENTION

In one aspect of the present invention, it is provided an endovascular device. The device comprises a metallic spiral coil and a coating on the coil; wherein the coating is formed either: on the outer surface of the spiral coil only such that the grooves of the coil remain uncoated and substantially free of the coating; or on the grooves of the coating such that the outer surface of the spiral coil remain uncoated and substantially free of the coating.

In some embodiments of the endovascular device, the metallic spiral coil comprises platinum, tungsten, titanium, silver, stainless steel, zirconium, or an alloy thereof. In some embodiments, the metallic spiral coil comprises Nitinol, polymers, or a biodegradable metal or alloy (e.g., magnesium or an alloy thereof).

In some embodiments of the endovascular device, the coating comprises a bioabsorbable polymer or a biodurable polymer. In some embodiments, the bioabsorbable polymer comprises a polyester polymer, e.g., polyglycolic acid (PGA), poly-L-lactic acid (PLLA), polycaprolactive, poly-L-lactide, polydioxanone, polycarbonates, polyanhydrides, polyglycolic acid/poly-L-lactic acid copolymers, and polyhydroxybutyrate/hydroxyvalerate copolymers, or combinations thereof. In some embodiments, the biodurable polymer comprises polyacrylate, polymethacrylate, polyether, or a fluorinated polymer. In some embodiments, the polymer can be polylactone, poly-alpha-hydroxy acids, poly(3-hydroxyalkanoates), polyglycols, polytyrosine carbonates, starch, gelatins, cellulose as well as blends and interpolymers containing these components. Examples of poly-alpha-hydroxy acids are polylactides, polyglycol acids, and their interpolymers. In some embodiments, the polymer can be caprolactone/glycolide copolymer or calcium stearoyl lactylate. Calcium stearoyl lactylate degrades into stearic and lactic acids. The polymer can also be acidic polyesters, such as a mixture of PLGA and hydroxyacetic acid (about equivalent molar ratios), or polyester anhydrides such as glycolic acid, lactic acid, or sebacic acid polymers.

In some embodiments of the endovascular device, the coating further comprises a bioactive agent.

In some embodiments of the endovascular device, the coating comprises a drug matrix layer comprising a bioactive agent, an optional primer layer underneath the drug matrix layer, and an optional a top layer immediately over the drug matrix layer, and wherein the optional top layer provides a controlled release of the bioactive agent.

In some embodiments of the endovascular device, the coating further comprises a biobeneficial material that enhances biocompatibility of the coating. Such biobeneficial material can be any material capable of enhancing biocompatibility of the coating. Examples of such biobeneficial material can be, e.g., a material that comprises choline, e.g., phosphoryl choline.

The various above embodiments of the endovascular device can be any endovascular device. In some embodiments, the device is a detachable aneurysm coil. In some embodiments, the endovascular device is a bare platinum coil.

In another aspect of the present invention, it is provided a method of forming a coating on an endovascular device. The device comprises a spiral coil body. The method comprises: forming a primary layer on the coil using a first solution comprising a primary layer material in a first solvent, removing the primary layer from the grooves of the spiral coil or the outer surface of the spiral coil, forming a second layer on the outer surface of the spiral coil or on the grooves of the spiral coil using a second solution comprising a second layer material and a second solvent, drying the second layer, removing the primary layer from the grooves of the spiral coil or the outer surface of the spiral coil, and drying the coating, wherein the primary layer material does not dissolve in the second solution and is not wet well by the second solution, and wherein the coating covers only the outer surface of the spiral coil or the grooves of the spiral coil.

Some embodiments of the method further comprise treating the coating with a solvent vapor to produce a smooth even coating.

In some embodiments of the method, optionally in combination with any or all of above various embodiments, an additional lubricant layer can be deposited on top of the second polymer layer, which imparts additional advantages or desirable properties to the coating, e.g., to prevent damage to the polymer layer during storage, to confer polymer integrity during deployment, and/or to decrease friction during deployment. In some embodiments, the lubricant layer can also contain pro-inflammatory factors embedded within the lubricant layer, or possess inherent pro-inflammatory properties.

In general any combination of solvents can be used for the first or second solvent as long as they do not mix together, which is shown by high interfacial tensions, and present disparate solubility parameters. In addition, the solvents must dissolve their respective polymers. The only first solvent we have tested was water. Second solvents that we have tested were: 1,2 Dichloroethane, 2-Phenoxyethanol, Acetone, Acetonitrile, Benzaldehyde, Benzonitrile, Benzyl alchohol, Chloroform, Dichloromethane, Dimethyl Adipate, Dimethyl sulfoxide, Dimethylformamide, Dioxane, Ethyl acetate, Hexafluoroisopropanol, Propylene carbonate. First and second solvents were chosen based on similar Hansen solubility parameters as the primary or secondary polymer, respectively. In some embodiments of the method, the first solvent is water, and the second solvent is chloroform.

In some embodiments of the method, optionally in combination with any or all of the various above embodiments, the primary layer material is dextran sulfate. Other materials for the primary layer material can be, e.g., polyethylene glycol, polyvinyl Alcohol, polyacrylic acid, polyvinylpyrrolidone, polyacrylamide, carboxymethyl cellulose, guar gum, hypromellose, glucose, polyvinylsulfate, polyvinyl phosphonic acid, mowiol, hydroxyethyl cellulose, dextran, dextran sulfate, glycolide, pullan, starch, xylan, polyallylamie, polyepoxysuccinic acid, amylose, galactan, cellulose, gelatin, pectin, chitosan. The second layer material comprises a bioabsorbable polymer or a biodurable polymer. In some embodiments, the bioabsorbabel polymer comprises a polyester, e.g., poly(lactic acid) (PLA), poly(lactic-co-glycolic acid) (PLGA), or a combination thereof. In some embodiments, the biodurable polymer comprises polyacrylate, polymethacrylate, polyether, or a fluorinated polymer. In some embodiments, the polymer can be polylactone, poly-alpha-hydroxy acids, poly(3-hydroxyalkanoates), polyglycols, polytyrosine carbonates, starch, gelatins, cellulose as well as blends and interpolymers containing these components. Exmaples of poly-alpha-hydroxy acids are polylactides, polyglycol acids, and their interpolymers. In some embodiments, the polymer can be caprolactone/glycolide copolymer or calcium stearoyl lactylate. Calcium stearoyl lactylate degrades into stearic and lactic acids.

In some embodiments of the method, optionally in combination with any or all of the above various embodiments, the second layer polymer comprises a pro-inflammatory factor or material that generates a transient and mild inflammation so as to accelerate wound healing. Examples of such pro-inflammatory materials are acidic polyesters are examples of pro-inflammatory coating materials that can accelerate healing. The polymer can also be acidic polyesters, such as a mixture of PLGA and hydroxyacetic acid (about equivalent molar ratios), or polyester anhydrides such as glycolic acid, lactic acid, or sebacic acid polymers. In some embodiments, where the second layer polymers are not inflammatory, the coating may contain fillers or particles that happen to cause transient and mild inflammation.

The various features of the spiral coil including the polymer, the coating, the layers of coating, and the bioactive agent are as described above or below.

In the various above embodiments of the method of invention, the endovascular device can be any endovascular device. In some embodiments, the device is a detachable aneurysm coil. In some embodiments, the endovascular device is a bare platinum coil.

In another aspect, it is provided a method of forming a coating on a spiral coil. The method comprises pre-stretching and without pre-stretching techniques such as rolling, spraying, stamping, printing, etc. Other coating techniques include: direct dip coating, roll coating, spray coating, and geometric printing.

In some embodiments of the method of making a spiral coil, optionally in combination with any or all of the above various embodiments, the method comprises an optional step. This step will precede all coating steps. This step pertains to direct modification of the metal surface such that it increases the adhesion of the polymer to the metal surface. This technique can be achieved by increasing the surface area of the spiral coil, or increase wetting of the polymer solution to the metal surface. Techniques to increase the surface area of the metal surface include: surface abrasion or acid etching. Techniques to increase the wetting of the polymer solution to the metal surface include plasma etching, plasma treatment, and surface cleaning.

In another aspect of the present invention, it is provided a method of treating or ameliorating a medical condition. The method comprises implanting in a mammalian subject an endovascular device according to any of the various embodiments described above or below. In some embodiments, the medical condition is intracranial aneurysm rupture.

### DETAILED DESCRIPTION OF THE INVENTION

In one aspect of the present invention, it is provided an endovascular device. The device comprises a metallic spiral coil and a coating on the coil; wherein the coating is formed either: on the outer surface of the spiral coil only such that the grooves of the coil remain uncoated and substantially free of the coating; or on the grooves of the coating only such that the outer surface of the spiral coil remain uncoated and substantially free of the coating.

In some embodiments of the endovascular device, the metallic spiral coil comprises platinum, tungsten, titanium, silver, stainless steel, zirconium, or an alloy thereof. In some embodiments, the metallic spiral coil comprises Nitinol, polymers, or a biodegradable metal or alloy (e.g., magnesium or an alloy thereof).

In some embodiments of the endovascular device, optionally in combination with any or all of the above various embodiments, the coating comprises a bioabsorbable polymer or a biodurable polymer. In some embodiments, the bioabsorbable polymer comprises a polyester polymer, e.g., polyglycolic acid (PGA), poly-L-lactic acid (PLLA), polycaprolactive, poly-L-lactide, polydioxanone, polycarbonates, polyanhydrides, polyglycolic acid/poly-L-lactic acid copolymers, and polyhydroxybutyrate/hydroxyvalerate copolymers, or combinations thereof. In some embodiments, the biodurable polymer comprises polyacrylate, polymethacrylate, polyether, or a fluorinated polymer. In some embodiments, the polymer can be polylactone, poly-alpha-hydroxy acids, poly(3-hydroxyalkanoates), polyglycols, polytyrosine carbonates, starch, gelatins, cellulose as well as blends and interpolymers containing these components. Exmaples of poly-alpha-hydroxy acids are polylactides, polyglycol acids, and their interpolymers. In some embodiments, the polymer can be caprolactone/glycolide copolymer or calcium stearoyl lactylate. Calcium stearoyl lactylate degrades into stearic and lactic acids. The polymer can also be acidic polyesters, such as a mixture of PLGA and hydroxyacetic acid (about equivalent molar ratios), or polyester anhydrides such as glycolic acid, lactic acid, or sebacic acid polymers.

In some embodiments of the endovascular device, optionally in combination with any or all of the above various embodiments, the coating further comprises a bioactive agent.

In some embodiments of the endovascular device, optionally in combination with any or all of the above various embodiments, the coating comprises a drug matrix layer comprising a bioactive agent, an optional primer layer underneath the drug matrix layer, and an optional a top layer immediately over the drug matrix layer, and wherein the optional top layer provides a controlled release of the bioactive agent.

In some embodiments of the endovascular device, optionally in combination with any or all of the above various embodiments, the coating further comprises a biobeneficial material that enhances biocompatibility of the coating. Such biobeneficial material can be any material capable of enhancing biocompatibility of the coating. Examples of such biobeneficial material can be, e.g., a material that comprises choline, e.g., phosphoryl choline.

The various above embodiments of the endovascular device can be any endovascular device. In some embodiments, the device is a detachable aneurysm coil. In some embodiments, the device is a bare platinum coil.

In another aspect of the present invention, it is provided a method of forming a coating on an endovascular device. The device comprises a spiral coil body. The method comprises: forming a primary layer on the coil using a first solution comprising a primary layer material in a first solvent, removing the primary layer from the grooves of the spiral coil or the outer surface of the spiral coil, forming a second layer on the outer surface of the spiral coil or on the grooves of the spiral coil using a second solution comprising a second layer material and a second solvent, drying the second layer, removing the primary layer from the grooves of the spiral coil or the outer surface of the spiral coil, and drying the coating, wherein the primary layer material does not dissolve in the second solution and is not wet well by the second solution, and wherein the coating covers only the outer surface of the spiral coil or the grooves of the spiral coil.

Some embodiments of the method further comprise treating the coating with a solvent vapor to produce a smooth even coating.

In some embodiments of the method, optionally in combination with any or all of the above various embodiments, an additional lubricant layer may be deposited on top of the second polymer layer, which imparts additional advantages or desirable properties to the coating, e.g., to prevent damage to the polymer layer during storage, to confer polymer integrity during deployment, and/or to decrease friction during deployment. In some embodiments, the lubricant layer can also contain pro-inflammatory factors embedded within the lubricant layer, or possess inherent pro-inflammatory properties.

In general any combination of solvents can be used for the first or second solvent as long as they do not mix together, which is shown by high interfacial tensions and present disparate solubility parameters. In addition, the solvents must dissolve their respective polymers. The only first solvent we have tested was water. Second solvents that we have tested were: 1,2 Dichloroethane, 2-Phenoxyethanol, Acetone, Acetonitrile, Benzaldehyde, Benzonitrile, Benzyl alchohol, Chloroform, Dichloromethane, Dimethyl Adipate, Dimethyl sulfoxide, Dimethylformamide, Dioxane, Ethyl acetate, Hexafluoroisopropanol, Propylene carbonate. First and second solvents were chosen based on similar Hansen solubility parameters as the primary or secondary polymer, respectively. In some embodiments of the method, the first solvent is water, and the second solvent is chloroform.

In some embodiments of the method, optionally in combination with any or all of the above various embodiments, the primary layer material is dextran sulfate. Other materials for the primary layer material can be, e.g., polyethylene glycol, polyvinyl Alcohol, polyacrylic acid, polyvinylpyrrolidone, polyacrylamide, carboxymethyl cellulose, guar gum, hypromellose, glucose, polyvinylsulfate, polyvinyl phosphonic acid, mowiol, hydroxyethyl cellulose, dextran, dextran sulfate, glycolide, pullan, starch, xylan, polyallylamie, polyepoxysuccinic acid, amylose, galactan, cellulose, gelatin, pectin, chitosan. The second layer material comprises a bioabsorbable polymer or a biodurable polymer. In some embodiments, the bioabsorbabel polymer comprises a polyester, e.g., poly(lactic acid) (PLA), poly(lactic-co-glycolic acid) (PLGA), or a combination thereof. In some embodiments, the biodurable polymer comprises polyacrylate, polymethacrylate, polyether, or a fluorinated polymer. In some embodiments, the polymer can be polylactone, poly-alpha-hydroxy acids, poly(3-hydroxyalkanoates), polyglycols, polytyrosine carbonates, starch, gelatins, cellulose as well as blends and interpolymers containing these components. Exmaples of poly-alpha-hydroxy acids are polylactides, polyglycol acids, and their interpolymers. In some embodiments, the polymer can be caprolactone/glycolide copolymer or calcium stearoyl lactylate. Calcium stearoyl lactylate degrades into stearic and lactic acids.

In some embodiments of the method, optionally in combination with any or all of the above various embodiments, the second layer polymer comprises a material that generates a transient and mild inflammation so as to accelerate wound healing. Examples of such pro-inflammatory coating materials are acidic polyesters are examples of pro-inflammatory coating materials that can accelerate healing. The polymer can also be acidic polyesters, such as a mixture of PLGA and hydroxyacetic acid (about equivalent molar ratios), or polyester anhydrides such as glycolic acid, lactic acid, or sebacic acid polymers. In some embodiments, where the second layer polymers are not inflammatory, the coating may contain fillers or particles that happen to cause transient and mild inflammation.

In the method of invention, the various features of the spiral coil including the polymer, the coating, the layers of coating, and the bioactive agent are as described above or below.

In the method of invention, the various above embodiments of the endovascular device can be any endovascular device. In some embodiments, the device is a detachable aneurysm coil. In some embodiments, the device is a bare platinum coil.

In another aspect, it is provided a method of forming a coating on a spiral coil. The method comprises pre-stretching and without pre-stretching techniques such as rolling, spraying, stamping, printing, etc. Other coating techniques include: direct dip coating, roll coating, spray coating, and geometric printing. All of these techniques—including the technique described above and below—may require the spiral coil to be stretched along the coil axis, prior to the coating methods, to expose the grooves such that the final coating is deposited exclusively on the coil surface.

Information on exemplary alternative coating techniques is provided below:

Direct dip coating—a spiral coil is immersed in a polymer solution (with appropriate solvent), withdrawn from the solution, and allowed to dry.

Roll coating—bioactive polymer is applied to a flat rubber stamping device. The bioactive polymer is applied to the spiral coil by touching the rubber stamp to an elongated spiral coil. The rubber stamp moves linearly along the coil, such that it rolls the coil. During this motion, the polymer releases from the rubber stamp, and is applied to the spiral coil.

Spray coating—a solution of bio active polymer is prepared and is deposited onto the spiral coil surface by atomization. This process is similar to airbrushing or spray painting.

In some embodiments of the method of making a spiral coil, optionally in combination with any or all of the above various embodiments, the method comprises an optional step. This step will precede all coating steps. This step pertains to direct modification of the metal surface such that it increases the adhesion of the polymer to the metal surface. This technique can be achieved by increasing the surface area of the spiral coil, or increase wetting of the polymer solution to the metal surface. Techniques to increase the surface area of the metal surface include: surface abrasion or acid etching. Techniques to increase the wetting of the polymer solution to the metal surface include plasma etching, plasma treatment, and surface cleaning.

In another aspect of the present invention, it is provided a method of treating or ameliorating a medical condition. The method comprises implanting in a mammalian subject an endovascular device according to any of the various embodiments described above or below. In some embodiments, the medical condition is intracranial aneurysm rupture.

The present invention is advantageous in that it allows the modification of bare metallic coils (e.g., bare platinum coils) such that only selected surfaces along the spiral coil is coated with a polymer. This polymer coating can be bioactive active, or may release a bioactive agent, or it may react with the local environment to provide bulking function. By leaving the grooves between each coil segment uncoated, the coating preserves the mechanical flexibility of the coil. Alternatively, when delivery of a bioactive agent is desired and the size of coil diameter is of concern, the present invention provides for coating only the grooves between the coil segments, thus delivering bioactive agents without increasing the overall diameter of the coil. The present invention can be applied to any currently available coil systems for the treatment of any medical condition that can be treated by an endovascular coil. An example of such medical conditions is brain aneurysm. For example, currently, the maximum diameter of the coil material that can be delivered through the microcatheter for intracranial aneurysm treatment is 0.018 inch that is known to provide the best mechanical support to resist the pulsatile blood flow. However, there is no coil material of this size that carries additional bioactivity (e.g., bioactivity imparted by a bioactive agent). The present invention will allow the coil material or system to have additional bioactive coating without impeding its mechanical property. Relatively large aneurysms will be treated more effectively so as to achieve less recanalization rate and improved treatment rate.

Additionally, the endovascular device provided herein is capable of generating a transient and mild inflammation condition at a site receiving the device or the surrounding area. A transient and mild inflammation condition can facilitate healing of wound of a site receiving a device of invention. In some embodiments, acid polyesters can be coated onto a device disclosed herein to generate transient and mild inflammation at the site receiving the device.

### Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. The practice of the present invention will employ, unless otherwise indicated, conventional methods of protein chemistry, biochemistry, and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Remington's Pharmaceutical Sciences, 18th Edition (Easton, Pa.: Mack Publishing Company, 1990).

All publications, patents and patent applications cited herein, whether supra or infra, are hereby incorporated by reference in their entirety.

The terms “effective amount” or “pharmaceutically effective amount” refer to a nontoxic but sufficient amount of the agent to provide the desired biological result. That result can be reduction and/or alleviation of the signs, symptoms, or causes of a disease, or any other desired alteration of a biological system. For example, an “effective amount” for therapeutic uses is the amount of the composition comprising a drug disclosed herein required to provide a clinically significant modulation in the symptoms associated with vascular permeability. An appropriate “effective amount” in any individual case may be determined by one of ordinary skill in the art using routine experimentation.

As used herein, the terms “treat” or “treatment” are used interchangeably and are meant to indicate a postponement of development of a disease associated with vascular permeability and/or a reduction in the severity of such symptoms that will or are expected to develop. The terms further include ameliorating existing symptoms, preventing additional symptoms, and ameliorating or preventing the underlying metabolic causes of symptoms.

The term “polymer” is defined as being inclusive of homopolymers, copolymers, and oligomers. The term “homopolymer” refers to a polymer derived from a single species of monomer. The term “copolymer” refers to a polymer derived from more than one species of monomer, including copolymers that may be obtained by copolymerization of two monomer species, those that may be obtained from three monomers species (“terpolymers”), those that may be obtained from four monomers species (“quaterpolymers”), etc. Some examples of polymers are bioabsorbable polymers and biodurable polymers. Further, as used herein, the term “polymer” includes any polymers that either directly, or indirectly by their degradation products will promote at least 25% increase in activities of neutrophils, macrophages, or other lymphocytes. Generally, such polymers do not include a polymer that tends to stick to itself when wet, as this would cause coil-coil friction during deployment and retrieval.

In some embodiments, the bioabsorbable polymer comprises a polyester, e.g., poly(lactic acid) (PLA), poly(lactic-co-glycolic acid) (PLGA), or a combination thereof or poly polyorthoesters. Bioabsorbable polymers can have acid, base, hydroxyl, or ester functional groups as side groups (pendant groups) or at one or both ends of the polymer backbone, which can also be referred to as acid-terminated, base-terminated, hydroxyl terminated, or ester terminated polymer. These polymers can be readily prepared according to established methodologies of polyester preparation. For example, acid terminated polyester can be readily prepared by using a diacid as the initiator in the preparation of the polyester. Likewise, amine-terminated (base-terminated), hydroxyl-terminated or ester-terminated polyester polymers can be readily prepared using a diamine, diol or an ester having a free hydroxyl group initiator in the preparation of the bioabsorbable polymer (e.g., PLA, PLGA, polyorthoester), respectively.

In some embodiments, the bioabsorbable polymer includes polymers that break down into acidic/basic monomers (e.g., PLA or polyorthoesters). The degradation products of these polymers can cause slightly inflammatory reaction.

In some embodiments, the biodurable polymer comprises polyacrylate, polymethacrylate, polyether, or a fluorinated polymer.

The term “poly(lactic acid-co-glycolic acid)” or “PLGA” refers to a copolymer formed by co-polycondensation of lactic acid, HO—CH(CH₃)—COOH, and glycolic acid, HO—CH₂—COOH.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender.

By “substantially free” is meant that at least 80% or more (e.g., 90% or more, 95% or more, or 99%) area of the grooves of the spiral coil remain uncoated. Conversely, in some embodiments, by “substantially free” is meant that at least 80% or more (e.g., 90% or more, 95% or more, or 99%) area of the outer surface of the spiral coil remains uncoated.

By “does not dissolve” is meant the primary layer material has a solubility in the second solvent of lower than 1 g /100 cc.

By “is wet not well” is meant the primary layer and the second solution has a contact angle (θ) that is 90° or larger (θ≧90°). The contact angle is the angle at which the liquid-vapor interface meets the solid-liquid interface. The contact angle is determined by the resultant between adhesive and cohesive forces. As the tendency of a drop to spread out over a flat, solid surface increases, the contact angle decreases. Thus, the contact angle provides an inverse measure of wettability. Adhesive forces between a liquid and solid cause a liquid drop to spread across the surface. Cohesive forces within the liquid cause the drop to ball up and avoid contact with the surface.

As used herein, the term “bioactive agent” can be any biologically active molecule. Any biologically active substance can be used as the source of biologically active molecules. Representative examples include laminin and growth factors such as IGF (insulin-like growth factors), TGF (transforming growth factors), FGB (fibroblast growth factors), including b-FGF (basic fibroblast growth factors), EGF (epidermal growth factors), VEGF (vascular endothelial growth factors), BMP (bone morphogenic proteins), PDGF (platelet-derived growth factors), or combinations thereof. These growth factors are well known and are commercially available.

The term “coil” can be any type of coil known in the art, such as, for example, a Guglielmi detachable coil (GDC). The coil can be coated with an absorbable polymeric material to improve long-term anatomic results in the endovascular treatment of intracranial aneurysms. The coil can further be coated to decrease friction to decrease the granulation tissue formation around the coils. In one aspect of the invention, the coat comprises at least one biocompatible and bioabsorbable polymer and growth factors, and is used to accelerate histopathologic transformation of unorganized clot into fibrous connective tissue in aneurysms.

As used herein, the term “solvent vapor” generally refers to the vapor of a volatile solvent capable of dissolving a polymer for forming the second layer of a coating disclosed herein. The volatile solvent can be the same as or different from the solvent for the second solution for forming the second layer of coating. An example of the volatile solvent is acetone. Another example of the volatile solvent is ethyl acetate.

As used herein, the term “transient and mild inflammation” refers to an inflammatory condition limited to the site of tissue receiving a device disclosed herein and the surrounding area that would disappear or clear in a short period of time, e.g., hours or days. Such transient and mild inflammation is within the knowledge of a medical practitioner or researcher and can be measured by, e.g., a slight elevation of temperature (e.g., an increase of temperature of 0.5 F, 1 F, 1.5 F, or 2 F) at the site of tissue receiving the device and the surrounding area.

### EXAMPLES

The following examples are illustrative and not limiting.

### Example 1

### Forming a Coating on Platinum/Tungsten Coils

The first step is performed by immersing the entire coil in an aqueous solution of dextran sulfate to form a primary layer, and then drawing the coil through a small aperture in a Teflon tape at controlled draw velocity to remove excess dextran sulfate. This first step confines the primary layer to the grooves of the coil. This dextran-coated coil is subsequently immersed and drawn from a polymer/chloroform solution (e.g., acid modified PLGA). The dextran sulfate can be replaced by any other polymer that does not dissolve in the second solution, and is not wet well by the second solution. After the PLGA is dried, the coil is immersed in water to remove the primary layer, and then dried. The coil is then flexed to remove any PLGA that has spanned over the grooves. Lastly the coil is exposed to acetone vapor to produce a smooth even coating which only covers the outer coil surface.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1-40. (canceled)

41. A method of forming a coating on an endovascular coil having a plurality of coil segments each separated by grooves, the method comprising:
providing a solution comprising a polymeric coating;
stretching the coil to expose the grooves;
depositing the polymeric coating on the coil surface such that the grooves of the coil remain substantially free of coating.

42. A method as recited in claim 41, wherein the coil comprises a metallic coil.

43. A method as recited in claim 41, wherein the metallic coil comprises platinum, tungsten, titanium, silver, stainless steel, zirconium, or an alloy thereof.

44. A method as recited in claim 41, wherein the coil comprises a coil axis, and wherein the coil is stretched along the coil axis to expose the grooves.

45. A method as recited in claim 41, wherein the coating comprises a bioabsorbable polymer or a biodurable polymer.

46. A method as recited in claim 45, wherein the coating further comprises a bioactive agent.

47. A method as recited in claim 45, further comprising:
depositing a layer of lubricant over the polymeric coating.

48. A method as recited in claim 41, wherein the coil comprises a Guglielmi Detachable Coil (GDC).

49. An endovascular device, comprising:
a metallic spiral coil comprising a plurality of coil segments each separated by grooves;
a polymeric coating disposed on an external surface of the coil segments;
wherein the grooves of the coil are substantially free of the polymeric coating.

50. A device as recited in claim 49, wherein the coil comprises a metallic coil.

51. A device as recited in claim 49, wherein the metallic coil comprises platinum, tungsten, titanium, silver, stainless steel, zirconium, or an alloy thereof.

52. A device as recited in claim 49, wherein the coating comprises a bioabsorbable polymer or a biodurable polymer.

53. A device as recited in claim 49, wherein the coating further comprises a bioactive agent.

54. A device as recited in claim 49, further comprising:
layer of lubricant over the polymeric coating.

55. A device as recited in claim 49, wherein the coil comprises a Guglielmi Detachable Coil (GDC).
